(19) United States
(12) Patent Application Publication
(10) Pub. No.: US 2010/0143481 A1
(43) Pub. Date: Jun. 10, 2010

(54) METHOD OF PREPARING SOLID DOSAGE FORMS OF MULTI-PHASIC PHARMACEUTICAL COMPOSITIONS

(76) Inventors: Dinesh Shenoy, Karnataka state (IN); Robert Lee, Boyertown, PA (US); Kumaresh Soppimath, Claremont, CA (US); Guru Betageri, Chino Hills, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/513,203

(22) PCT Filed: Nov. 8, 2007
(86) PCT No.: PCT/US07/84141

§ 371 Date: Feb. 12, 2010

Related U.S. Application Data
(60) Provisional application No. 60/857,511, filed on Nov. 8, 2006.

(51) Int. Cl. A61K 9/14; A61K 47/02; A61K 47/38; A61K 47/34; A61K 47/32; A61K 47/30; A61K 47/36; A61K 47/44; A61P 43/00

(52) U.S. Cl. 424/489; 514/770; 514/781; 514/772.3; 514/772.6; 514/772.4; 514/769; 514/777; 514/778; 514/786

(57) ABSTRACT

Pharmaceutical formulations comprising a multi-phasic pharmaceutical composition, and an adsorbent carrier, where the pharmaceutical formulation is a solid dosage form. Methods for preparing such pharmaceutical compositions are described.

### CROSS-REFERENCE TO RELATED APPLICATIONS

This invention claims priority to U.S. Provisional Application No. 60/857,511 filed Nov. 8, 2006, the disclosure of which is incorporated herein by reference in its entirety.

### FIELD OF THE INVENTION

Generally, the present invention is directed to solid form pharmaceutical dosages. In particular, the invention is directed to the preparation of and use of multi-phasic pharmaceutical compositions in a solid dosage form.

The following U.S. patent applications are specifically incorporated by reference: U.S. patent application Ser. No. 11/714,274, filed on Mar. 6, 2007; and U.S. Patent Application No. 60/881,470, filed on Jan. 22, 2007.

### BACKGROUND

Liquid form drug compositions are ubiquitous throughout the pharmaceutical industry, existing as compositions of solutions, suspensions, emulsions, and the like. While liquid dosage forms are convenient forms, especially for pediatric and geriatric applications, conversion of these liquid compositions to a solid dosage form (i.e., tablets or capsules) can add significantly to both patient compliance and the commercial value to the products. Simple aqueous-based solutions or suspensions may be converted to a corresponding solid dosage form by lyophilizing with suitable cryoprotectants, the resulting mass being mixed with one or more suitable diluents, followed by filling into capsules or compressing into tablets.

Multi-phasic pharmaceutical compositions may contain a solubilized API, a particulate API, or a mixture of the two. Micellular nanoparticle (MNP) compositions are multi-phasic compositions comprised of a solubilized, emulsified, and/or solid particulate active pharmaceutical ingredient (API), variously known as nanoparticulate compositions. Delivery of such multi-phasic compositions has been described as being effected, for example, via cream or lotion, in which an API is administered to a subject. However, not all APIs are able to be effectively administered to a patient via such routes due to any of a number of obstacles such as, solubility of the API, long term stability of the API, reactivity of the API with other materials used in transdermal applications, skin tolerance to the API, and various other problems. Thus, other routes of administration of multi-phasic formulations of various APIs are needed.

### SUMMARY

In one aspect, a pharmaceutical formulation is provided comprising a multi-phasic pharmaceutical composition and an adsorbent carrier, wherein the pharmaceutical formulation is a solid dosage form. In some embodiments, the adsorbent carrier is a clay, a silicate, a cellulose-based polymer, microsponges, other synthetic polymers, or a mixture of any two or more thereof. In some embodiments, the pharmaceutical formulation further comprises a polymeric carrier, a phospholipid carrier, or a mixture of any two or more thereof. In other embodiments, the pharmaceutical formulations further comprise a lubricant, an antioxidant, a coloring agent, a flavoring agent, a preservative, a sweetener, a volatile oil, or a mixture of any two or more thereof.

In some embodiments, the pharmaceutical formulation is comprised within a capsule or tablet.

In some embodiments, the pharmaceutical formulation disintegrates to release an active pharmaceutical ingredient upon introduction to in an aqueous medium.

In some embodiments, the multi-phasic pharmaceutical composition comprises at least one active pharmaceutical ingredient, wherein the active pharmaceutical ingredient is in a particulate state and/or in a soluble state; a solvent; a non-miscible liquid; a stabilizer; and water.

In another aspect, methods are provided for the preparation of a pharmaceutical formulation comprising mixing an active pharmaceutical ingredient, a solvent, a stabilizer, and a non-miscible liquid to form a first mixture; emulsifying the first mixture with water to form a multi-phasic pharmaceutical composition; and mixing the emulsified first mixture with an adsorbent carrier to form a solid dosage form. The pharmaceutical formulation can comprise more than one active pharmaceutical ingredient. For example, combination pharmaceutical formulations can comprise two or more active pharmaceutical ingredients useful in treating a particular condition. Examples include, but are not limited to, lipid lowing agents such as a fibrate (e.g., fenofibrate) in combination with a statin.

Both the foregoing summary and the following brief description of the drawings and the detailed description are exemplary and explanatory and are intended to provide further details of the compositions and methods as claimed. Other objects, advantages, and novel features will be readily apparent to those skilled in the art from the following detailed description.

### BRIEF DESCRIPTION OF THE DRAWINGS

FIG. 1: Visually shows the disintegration and dissolution of four different formulations: Nova II tablet; Nova III tablet; Nova III granules, and MNP emulsion.

FIG. 2: Shows the release rate over time for a TRICOR® tablet disintegrated into a nanosuspension, wherein dissolution occurred within 4 min. and almost 100% of drug was released within 20-30 min.

FIG. 3: Shows the percent of API released over time for two different formulations; a tablet and an emulsion.

### DETAILED DESCRIPTION

### A. Definitions

The present invention is described herein using several definitions, as set forth below and throughout the application.

For the purposes of this disclosure and unless otherwise specified, “a” or “an” means “one or more.”

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

“Adsorbent carrier” refers to materials, usually solid, employed to adsorb and/or absorb a liquid formulation.

As used herein, the terms “capsules,” “tablets,” “lozenges,” and “cachets” are synonymous terms and are used interchangeably, any individual term representing the group, unless specifically noted that only a capsule, a tablet, a lozenge, or a cachet is envisioned for a particular purpose.

“Cellulose” includes the various forms of cellulose known for use in pharmaceutical formulations, including but not limited to, ethyl cellulose, cellulose acetate, carboxymethyl cellulose calcium, sodium carboxymethyl cellulose, methyl cellulose, hydroxyethyl cellulose, hydroxypropyl cellulose, hydroxypropyl methyl cellulose, hydroxypropylmethyl cellulose phthalate, microcrystalline cellulose, and mixtures thereof.

Croscarmellose sodium is cross-linked sodium carboxymethyl cellulose.

“Crospovidone” is a water-insoluble cross-linked homopolymer of 1-vinyl-2-pyrrolidinone.

“Cyclodextrin” refers to a family of cyclic oligosaccharides containing at least six D-(+)-glucopyranose units.

“Emulsifier,” as used herein, refers to a material that promotes the formation of an emulsion.

As used herein, the term “emulsion” refers to a dispersion of one non-miscible liquid in another liquid.

“Fatty acid,” as used herein, refers to any of the members of a large group of monobasic acids, especially those found in animal and vegetable fats and oils. In some embodiments the fatty acid is straight or branched chain alkyl or alkenyl group having 6 to 22 carbons, wherein the carboxylic acid is at one terminus of the carbon chain.

“Glycerides,” as used herein, refers to esters formed between one or more acids and glycerol. In some embodiments, the acids are fatty acids. Medium-chain glycerides are glycerol esters of medium-chain fatty acids containing from 6 to 12 carbon atoms, or, in some embodiments, 6 to 10 carbon atoms. Medium chain fatty acids include: caproic acid (C₆); caprylic acid (C₈), capric acid (C₁₀), and lauric acid (C₁₂). Long chain glycerides are glycerol esters of long chain fatty acids containing from 12 to 22 carbon atoms, or in some embodiments, 12 to 18 carbon atoms.

“Lipid,” as used herein, refers to any of a group of organic compounds, including, but not limited to the fats, oils, waxes, sterols, and triglycerides, that are insoluble in water but soluble in non-polar organic solvents, and are oily to the touch.

As used herein, “microsponge” refers to a porous material capable of adsorbing or absorbing liquids

As used herein, the term “non-miscible liquid” refers to a liquid that does not dissolve in another liquid. Non-miscible liquids are capable of forming emulsions.

“Particulate state,” as used herein, refers to insoluble particles of a given material.

“Phospholipid,” as used herein, refers to phosphorous-containing lipids that are composed mainly of fatty acids, a phosphate group, and a simple organic molecule, e.g. glycerol. Phospholipids may also be referred to as phosphatides.

Povidone, as used herein, is a polymer of 1-vinyl-2-pyrroldinone, and having a wide range of average molecular weight. In some embodiments, the povidone has an average molecular weight of from about 2,500 g/mol to about 300,000 g/mol, or greater.

As used herein, “solubilized state,” refers to a solution phase material, such as an API. Such solution phases include dissolution in a solvent, including water, or dissolution in one or more liquid components of an emulsion.

“Sorbitan,” as used herein, refers to dehydrated Sorbitol.

“Starch” refers to a complex carbohydrate consisting of amylase and amylopectin. “Pregelatinized starch” is starch that has been chemically and/or mechanically processed to rupture all or part of the granules in the presence of water and subsequently dried. Some types of pregelatinized starch may be modified to render them compressible and flowable in character.

“Sugar fatty acid,” as used herein, refers to a fatty acid with a sugar moiety attached.

The term “subject,” as used herein, refers to any animal that can experience the beneficial effects of the formulations and methods embodied herein. Preferably, the animal is a mammal, and in particular a human, although it is not intended to be so limited. Examples of other suitable animals include, but are not limited to, rats, mice, monkeys, dogs, cats, cattle, horses, pigs, sheep, and the like.

As used herein, the phrase “therapeutically effective amount” shall mean the drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

It will be readily understood by those of skill in the art, that some materials identified below as belonging to a category such as an adsorbent carrier, polymeric carriers, phospholipid carriers, pharmaceutically acceptable additives, or other carriers or additives may fall into one or more of those categories, although the material is listed in only one category. For example, magnesium aluminum silicate is both an adsorbent carrier and a synthetic or semi synthetic polymeric carrier. As another example, cellulose may be an adsorbent carrier and a polymeric carrier. Other such materials belonging in more than one category, but listed in only one category, will be readily identified by one of skill in the art.

### B. Solid Dosage Form Multi-phasic Compositions

Multi-phasic compositions are versatile vehicles for a wide variety of active pharmaceutical ingredients, and can be used for the delivery of poorly water-soluble compounds. For example, poorly water-soluble pharmaceuticals tend to be very difficult to deliver to a patient, however, multi-phasic compositions comprising both particulate state API and solubilized state API may provide a new route for oral, buccal, or rectal administration for such pharmaceuticals.

In one aspect, pharmaceutical formulations are provided comprising a multi-phasic pharmaceutical composition, and an adsorbent carrier, wherein the pharmaceutical formulation is a solid dosage form. In such pharmaceutical formulations, the multi-phasic pharmaceutical composition is preferably present at about 1 to about 90 wt %. The multi-phasic pharmaceutical composition can comprise two or more API. In one example, the multi-phasic pharmaceutical composition can comprise two or more active pharmaceutical ingredients useful in treating a particular condition. Examples include, but are not limited to, lipid lowing agents such as a fibrate (e.g., fenofibrate) in combination with a statin.

Some multi-phasic pharmaceutical compositions, known as MNPs, have been described previously. Multi-phasic pharmaceutical compositions of the present application comprise at least one active pharmaceutical ingredient; a solvent; a non-miscible liquid; a stabilizer; and water, wherein the active pharmaceutical ingredient is present in a particulate state, in a solubilized state, or in both a particulate and a solubilized state. Solid dose pharmaceutical formulations prepared from multi-phasic pharmaceutical compositions may be formulated into any suitable dosage form, such as a capsule or tablet. In some embodiments, the API is present in the solid dosage form at about 0.1 to about 70 wt %. When such pharmaceutical formulations are placed in aqueous media, the formulations disintegrate to release the active pharmaceutical ingredient. In some embodiments, the API is released in the form in which it existed in the multi-phasic pharmaceutical composition, i.e. in a particulate state and/or in a solubilized state.

In multi-phasic pharmaceutical compositions, when the API is present in both a particulate state and in a solubilized state, the amount of an API in a particulate state and the amount of an API in a solubilized state may vary. In some embodiments, the amount of API in the particulate state ranges from about 5 wt % to about 95 wt %, from about 10 wt % to about 90 wt %, from about 15 wt % to about 85 wt %, from about 20 wt % to about 80 wt %, from about 25 wt % to about 78 wt %, from about 30 wt % to about 75 wt %, from about 35 wt % to about 73 wt %, from about 40 wt % to about 70 wt %, from about 45 wt % to about 70 wt %, from about 50 wt % to about 70 wt %, from about 60 wt % to about 70 wt %, and/or from about 65 wt % to about 70 wt %. In some embodiments, the amount of API in the solubilized state ranges from about 0.5 wt % to about 80 wt %, from about 1.0 wt % to about 75 wt %, from about 5 wt % to about 70 wt %, from about 10 wt % to about 65 wt %, from about 15 wt % to about 60 wt %, from about 20 to about 55 wt %, from about 25 wt % to about 50 wt %, from about 25 wt % to about 45 wt %, from about 25 wt % to about 40 wt %, from about 28 wt % to about 35 wt %, and/or from about 28 wt % to about 33 wt %. The amount of API in a particulate state and the amount of API in a solubilized state for a multi-phasic composition may also be expressed as a weight ratio of the amount of API in a particulate state to the amount of API in the solubilized state. For example, such a ratio may range from about 95:5 to about 5:95. In some embodiments, the ratio is about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, or about 5:95.

Pharmaceutical formulations embodied herein comprise a multi-phasic pharmaceutical composition and an adsorbent carrier. Without being bound by theory, adsorbent carriers adsorb the non-miscible liquid (in some embodiments, an oil) that is present in the multi-phasic pharmaceutical composition to aid in the formation of a solid dosage form pharmaceutical formulation. Suitable adsorbent carriers for use in the embodied pharmaceutical formulations include porous materials, clays, silicates, cellulose-based polymers, microsponges, other synthetic polymers, or mixtures of any two or more thereof. Exemplary clays include attapulgite, bentonite, kaolin, perlite, talc, vermiculites, zeolites, or a mixture of any two or more thereof. Exemplary silicates include aluminum silicate, magnesium aluminum silicate, hydrous calcium silicate, colloidal silicon dioxide, magnesium aluminometasilicate, and mixtures of any two or more thereof. Exemplary cellulose-based polymers include carboxymethyl cellulose calcium, carboxymethyl cellulose sodium, cellulose, cellulose acetate, cellulose acetate phthalate, ethyl cellulose, hydroxyethyl cellulose, hydroxypropyl cellulose, hydroxypropyl methylcellulose, hydroxypropyl methylcellulose phthalate, methylcellulose, microcrystalline cellulose, powdered cellulose, or a mixture of any two or more thereof. Other synthetic polymers suitable for use as adsorbent carriers include cross-linked acrylic polymers, polypropylene, polyurethane foams, or mixtures of any two or more thereof.

Other adsorbent carriers that may be used in the embodied pharmaceutical formulations include, but are not limited to, calcium carbonate, calcium phosphate dibasic anhydrous, calcium phosphate dibasic dehydrate, calcium phosphate tribasic, calcium sulfate, lactose, magnesium carbonate, magnesium oxide, mannitol, silicon dioxide, sodium starch glycolate, sodium chloride, sorbitol, starch, sucrose, or a mixture of any two or more thereof.

Other carriers and additives may also be included in the embodied pharmaceutical formulations. Such other carriers and additives may be used to give binding, coloring, compressing, filling, flavoring, lubricating, and/or preserving properties to the pharmaceutical formulations or they may be used for other purposes known to those of skill in the art. For example, other carriers and additives may include, but are not limited to polymeric carriers, phospholipid carriers, lubricants, antioxidants, coloring agents, flavoring agents, preservatives, sweeteners, volatile oils, and/or a mixture of any two or more thereof.

Exemplary polymeric carriers that may be used in the embodied pharmaceutical formulations include, but are not limited to, carbomers, croscarmellose sodium, crospovidone, cyclodextrins, β-cyclodextrins, ducosate sodium, hydroxypropyl-β-cyclodextrins, γ-cyclodextrins, polyanionic-β-cyclodextrins, sulfobutylether-7-β-cyclodextrin, methacrylic acid copolymers, poloxamer, polydextrose, polyethylene oxide, polymethacrylate polymers, poly(methacrylic acid-methyl methacrylate), poly(methacrylic acid-ethyl acrylate), ammonio methacrylate copolymer, poly(ethyl acrylate-methylmethacrylate-trimethylammonioethyl methacrylate chloride), poly(ethyl acrylate-methyl methacrylate), polysaccharides, polyvinyl alcohol with an average molecular weight of from about 20,000 to about 200,000 g/mol, polyvinylpyrrolidine/vinylacetate, povidone with an average molecular weight of from about 2,500 to about 300,000 g/mol, poloxamer, sodium starch glycolate, or a mixture of any two or more thereof. Exemplary polysaccharides include, but are not limited to, acacia, alginic acid, carrageenan, ceratonia, chitosan, compressible sugar, confectioner's sugar, confectioner's sugar, dextrates, dextrates, dextrin, dextrin, dextrose, dextrose, fructose, fumaric acid, gelatin, glucose, liquid, glyceryl behenate, guar gum, lactitol, lactose, maltodextrin, maltodextrin, maltose, maltose, mannitol, polydextrose, polymethacrylates, pregelatinized starch, sodium alginate, sodium alginate, sorbitol, starch, pregelatinized starch, sterilizable maize, sucrose, sucrose, sugar spheres, tragacanth, trehalose, xylitol, or a mixture of any two or more thereof.

Some the polymeric carriers may also be variously known in the art as disintegrants, compression aids, or binders. For example, disintegrants may include, but are not limited to, cellulose-based polymers; polysaccharides; other materials such as croscarmellose sodium, crospovidone, docusate sodium, magnesium aluminum silicate, colloidal silicon dioxide, calcium phosphate tribasic, povidone; or a mixture of any two or more thereof, as well as other materials and mixtures known to those of skill in the art to be useful as disintegrants. Compression aids may include, but are not limited to, polysaccharides and cellulose-based polymers and also non-polymeric materials such as inorganic salts, including but not limited to, calcium carbonate, calcium phosphate, calcium sulfate, magnesium carbonate, magnesium oxide, sodium chloride. Binders may also include materials such as polysaccharides and other synthetic or semi-synthetic polymers.

Exemplary phospholipid carriers that may be used in the embodied pharmaceutical formulations include, but are not limited to, diphosphatidylglycerol, glycolipids, phosphatidic acid, phosphatidylcholine, phosphatidylethanolamine, phosphatidylglycerol, phosphatidylinositol, phosphatidylserine, sphingomyelin, or a mixture of any two or more thereof. Exemplary lubricants include magnesium stearate, talc, stearic acid, calcium stearate, zinc stearate, glyceryl palmitostearate, glyceryl behenate, light mineral oil, micronized poloxamers, polyethylene glycol, l-leucine, vegetable oil.

The pharmaceutical formulations embodied herein may also include, but are not limited to, pharmaceutically acceptable additives such as an antioxidant, a coloring agent, a flavoring agent, a preservative, a sweetener, a volatile oil, or a mixture of any two or more thereof. Exemplary antioxidants include, but are not limited to, ascorbic acid, ascorbyl palmitate, butylated hydroxyanisole, butylated hydroxytoluene, ethylenediaminetetraacetic acid, salts of ethylenediaminetetraacetic acid, propyl gallate, sodium metabisulfite, vitamin E, esters of vitamin E, or a mixture of any two or more thereof. Exemplary preservatives include, but are not limited to, butylparaben, calcium sorbate, ethylparaben, methylparaben, monothioglycerol, potassium sorbate, propylparaben, sodium benzoate, sodium sorbate, sorbic acid, or a mixture of any two or more thereof. Exemplary sweeteners include, but are not limited to, aspartame, glycyrrhizin salts, monoammonium glycyrrhizinate, saccharin, saccharin calcium, saccharin sodium, sugar, sucralose, or a mixture of any two or more thereof. Exemplary flavoring agents include, but are not limited to, anise, banana, cherry, chocolate, citric acid, lemon, menthol, orange, peppermint, pineapple, rum, sodium citrate, strawberry, vanillin, ethyl vanillin, or a mixture of any two or more thereof. Exemplary coloring agents include, but are not limited to, FD&C blue #1, FD&C blue #2, FD&C green #3, FD&C red #3, FD&C red #4, FD&C yellow #5, FD&C yellow #6, D&C blue #4, D&C green #5, D&C green #6, D&C orange #4, D&C orange #5, iron oxides, or a mixture of any two or more thereof. Exemplary volatile oils include, but are not limited to, balm oil, bay oil, bergamot oil, cedarwood oil, cherry oil, cinnamon oil, clove oil, origanum oil, peppermint oil, or a mixture of any two or more thereof.

The use of solid dosage forms such as capsules, tablets, lozenges, and/or cachets is well known in the art for the oral, buccal, or rectal administration of a pharmaceutical agent to a subject. The pharmaceutical formulations embodied herein, may be used in the preparation of such capsules, tablets, lozenges, and/or cachets. Capsules may be hard or soft, and may be made of a variety of materials known to those of skill in the art, including, but not limited to, cellulose materials, gelatin, carrageenan, agar, and pectin.

Active pharmaceutical ingredients useful in the embodied multi-phasic pharmaceutical compositions include any suitable API for multi-phasic compositions. For example, suitable APIs may include, but are not limited to agents used in the treatment of AIDS, agents used in treatment of heart disorders, analgesics, anesthetics, anorexiants, anthelmintics, anti-allergic agents, anti-anginal agents, antiarrhythmic agents, anticholinergics, anticoagulants, antidepressants, antidiabetic agents, antidiuretic agents, anti-emetic agents, antiepileptics, anti-fungals, antihistamines, anti-hypertensive agents, anti-inflammatory agents, anti-migraine agents, antimuscarinic agents, antimycobacterial agents, antineoplastic agents including, antiparkinsonian agents, antithyroid agents, antiviral agents, astringents, blocking agents, blood products, blood substitutes, cardiac inotropic agents, cardiovascular agents, central nervous system agents, chelating agents, chemotherapy agents, colony stimulating factors, corticosteroids, cough suppressants, dermatological agents, diuretics, dopaminergics, elastase inhibitors, endocrine agents, ergot alkaloids, expectorants, gastrointestinal agents, genitounnary agents, growth hormone releasing hormone, growth hormones, hematological agents, hematopoietic agents, hemostatics, hormones, immunologic agents, immunosuppressants, interleukins, interleukin analogues, lipid regulating agents, luteinizing hormone releasing hormone, muscle relaxants, narcotic antagonists, nutrients, nutritional agents, oncology therapies, organic nitrates, parasympathomimetics, prostaglandins antibiotics, renal agents, respiratory agents, sedatives, sex hormones, stimulants, sympathomimetics, systemic anti-infectives, tactolimuls, thrombolytic agents, thyroid agents, treatments for attention deficit disorder, uterine-active agents, vaccines, vasodilators, xanthines, or mixtures of any two or more thereof. Specific examples of API will be readily recognized by one of skill in the art, and may include, but are not limited to, raloxifene, an antiviral compound such as acyclovir, a compound useful in the relief of symptoms associated with perennial and seasonal allergic rhinitis; vasomotor rhinitis; allergic conjunctivitis; mild, uncomplicated urticaria and angioedema; or the amelioration of allergic reactions to blood or plasma; or dermatographism or as adjunctive therapy in anaphylactic reactions. Examples of such compounds include, but are not limited to, loratidine, desloratidine, and cetirizine. In one embodiment, the active pharmaceutical ingredient is acyclovir, an immunosuppressant such as cyclosporine or sirolimus, naltrexone, alendronic acid, ceterizine, nicotine, testosterone, progesterone, or estradiol.

In one embodiment of the invention, the multi-phasic pharmaceutical compositions are suitable for delivery of poorly water soluble drugs. As defined herein, “poorly water soluble” drugs have a solubility in water or another media of less than about 30 mg/mL, less than about 20 mg/mL, or less than about 10 mg/mL.

Solvents useful in the embodied pharmaceutical formulations include, but are not limited to, an alcohol, N-methylpyrrolidinone, methoxypolyethylene glycol, polyethylene glycol, polyethylene oxide, ethoxy diglycol, triacetin, dimethyl sulfoxide, propylene glycol, isopropyl myristate, mono-, di- or tri-glycerides, or a mixture of any two or more thereof. Exemplary alcohols include benzyl alcohol, ethyl alcohol, methyl alcohol, or a mixture of any two or more thereof. Exemplary polyethylene glycols have an average molecular weight of about 1000 g/mol or greater, and the methoxypolyethylene glycol has an average molecular weight of about 1000 g/mol or greater. In other embodiments, the polyethylene glycol has an average molecular weight of from about 1000 g/mol to about 20,000 g/mol, and the methoxypolyethylene glycol has an average molecular weight of from about 1000 g/mol to about 20,000 g/mol.

Non-miscible liquids for use in the embodied pharmaceutical formulations include, but are not limited to, fatty acids, medium chain glycerides, long chain glycerides, ethyl esters of a fatty acid, propylene glycol fatty acid esters, sorbitan fatty acid esters, polyglyceryl fatty acid esters, glyceryl mono-, di-, or tri-caprylic acid esters; glyceryl mono-, di-, or tri-capric acid esters; or a mixture of any two or more thereof. Non-miscible liquids also include vegetable oils, nut oils, fish oils, lard oil, mineral oils, squalane, tricaprylin (1,2,3-trioctanoyl glycerol), and mixtures of any two or more thereof. For example, almond oil (sweet), apricot seed oil, borage oil, canola oil, coconut oil, corn oil, cotton seed oil, fish oil, jojoba bean oil, lard oil, linseed oil (boiled), macadamia nut oil, medium chain triglycerides, mineral oil, olive oil, peanut oil, safflower oil, sesame oil, soybean oil, sunflower seed oil, wheat germ oil, mineral oil (light), DL-α-tocopherol, ethyl oleate, ethyl linoleate, glyceryl behenate, glyceryl monooleate, glyceryl monostearate, glyceryl palmitostearate, linoleic acid, linolenic acid, oleic acid, palmitostearic acid, peppermint oil, polyglyceryl oleate, propylene glycol monolaureate, propylene glycol dilaureate, sorbitan monolaurate, sorbitan monooleate, sorbitan monopalmitate, sorbitan monostearate, sorbitan trioleate, stearic acid, tetraglyceryl monooleate, or a mixture of any two or more thereof are all examples of non-miscible liquids for use in the embodied pharmaceutical formulations.

Stabilizers useful in the embodied pharmaceutical formulations include, but are not limited to, non-phospholipid surfactants, non-phenol polyethylene glycol ethers, sorbitan esters, polyethylene glycol esters, block polymers, acrylic polymers, ethoxylated fatty acids, ethoxylated alcohols, ethoxylated fatty acid esters, monoglycerides, silicon-based surfactants, polysorbates, tergitols, sugar fatty acid ester; a sucrose mono-, di-, or tri-fatty acid ester; a polyoxyethylene castor oil compound; a polyoxyethylene sorbitan fatty acid ester; a polyoxyethylene mono- or di-fatty acid ester; a polyoxyethylene alkyl ether; a glyceryl mono-, di-, or tri-fatty acid ester; a mixtures of polyoxyethylene mono- or di-ester of a C₈-C₂₂ fatty acid; a glyceryl mono-, di-, or tri-ester of a C₈-C₂₂ fatty acid, or a mixture of any two or more thereof. For example, the stabilizer may be ARLACEL™, BRIJ™, Cremophore RH-40, glycerin monostearate, PEMULEN™, Pluronics™, polyethylene glycol stearate, polyoxyl 35 castor oil, polyoxyl 40 hydrogenated castor oil, polyoxyl 60 hydrogenated castor oil, polysorbate 20, polysorbate 40, polysorbate 60, polysorbate 80, polyoxyl 40 stearate, polyoxyl 40 oleate, polyoxyl 20 cetostearyl ether, polyoxyl 10 oleyl ether, sodium dioctyl sulfosuccinate, sodium lauryl sulfate, SPAN™, TERGITOL™ NP-40, TERGITOL™ NP-70, DL-α-tocopheryl polyethylene glycol succinate, TWEEN™ 20, TWEEN™ 60, TWEEN™ 80, or a mixture of any two or more thereof.

Methods of preparing the pharmaceutical formulations are also provided. Such methods comprise mixing an active pharmaceutical ingredient a solvent, a stabilizer, and a non-miscible liquid to form a first mixture; emulsifying the first mixture with water to form a multi-phasic pharmaceutical composition; and mixing the emulsified first mixture with an adsorbent carrier to form a solid dosage form. The methods may further comprise pressing the solid dosage form into a capsule or tablet. In such embodied methods, the API may be present at about 0.1 to about 70 wt % of the capsule or tablet.

In some embodied methods, the multi-phasic composition comprises globules of the non-miscible liquid and the globules have a diameter of less than about 10 μm. For example, the globules may have a diameter of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

In some embodied methods, the multi-phasic composition comprises at least a portion of the API in particulate form. In some embodiments, the average diameter of the particles of the particulate form is from about 1 nm to about 10 microns. In some embodiments, the average diameter of the particles of the particulate form is less than about 10 microns. For example, the average diameter of the particles may be less than about 9 microns, less than about 8 microns, less than about 7 microns, less than about 6 microns, less than about 5 microns, less than about 4 microns, less than about 3 microns, less than about 2 microns, or about 1 micron or greater. In other embodiments, the average diameter of the particles is less than about 1 micron, such as from about 1 nm to about 1 micron. For example, the diameter of the API particles may be less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

One skilled in the art will readily realize that all ranges and ratios discussed can and do necessarily also describe all subranges and subratios therein for all purposes and that all such subranges and subratios also form part and parcel of this invention. Any listed range or ratio can be easily recognized as sufficiently describing and enabling the same range or ratio being broken down into at least equal halves, thirds, quarters, fifths, tenths, etc. As a non-limiting example, each range or ratio discussed herein can be readily broken down into a lower third, middle third and upper third, etc.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention. All publicly available documents referenced herein, including but not limited to US patents, are specifically incorporated by reference.

### EXAMPLES

### Example 1

A multi-phasic composition was first prepared as a placebo composition (i.e., without any API). Ethyl alcohol (8.8 wt %) was mixed with polysorbate 80 (9.4 wt %) and soybean oil (50.2 wt %). Water (31.6 wt %) was added and the resulting composition was subjected to emulsification using a paddle-type stirrer. The emulsion was processed using a high-pressure homogenizer (APV-1000) operating at 10,000 psi by passing through the homogenizer three times.

An API may be incorporated in the above preparation. The API may be: (i) completely soluble, (ii) partially soluble, or (iii) completely insoluble in the vehicle. Once dispersed in a multi-phasic composition, the API is preferably present in both a solubilized and a particulate state.

Using the multi-phasic composition from above, a tablet formation (i.e. solid dosage form) was prepared. The placebo product above was mixed with magnesium aluminometasilicate (Neusilin US2, particle size 80 μm) in a weight ratio of 1:1. This formed the multi-phasic premix which was subject to further granulation as described below.

The multi-phasic premix (8 g), microcrystalline cellulose (1.2 g, Avicel PH-103), and cross-linked polyvinylpyrrolidinone (0.3 g, crospovidone, Polyplasdone XL) were mixed uniformly by geometric mixing. The powder was granulated with an aqueous solution of polyvinylpyrrolidinone (povidone, Kollidon 30, 0.5 g in 8 g of water) as a granulating solution to obtain a pre-granular mass. The pre-granular mass was dried at 40° C. for 1.5 hr. After drying, granules were passed through a #30 sieve (˜500 μm hole size) and mixed with Polyplasdone XL (0.2 mg) and magnesium stearate (100 mg). Tablets (diameter=0.9 cm) were compressed using an automatic compression machine (Riddhi mini-press, model RDB4-10) with a target weight for the final solid dosage form of 250 mg.

### Example 2

The purpose of this example was to develop a tablet dosage form for a fenofibrate emulsion.

### A. Description and Evaluation of Tricor®.

Tricor® tablets are film coated yellow colored oval shaped tablets weighing about ˜0.216 g, and containing 48 mg of fenofibrate. Each tablet contains hypromellose 2910 (3 cps), docusate sodium, sucrose, sodium lauryl sulfate, lactose monohydrate, silicified microcrystalline cellulose, crospovidone, and magnesium stearate. In addition, individual tablets contain: (a) 48 mg tablets: polyvinyl alcohol, titanium dioxide, talc, soybean lecithin, xanthan gum, D&C Yellow #10 aluminum lake, FD&C Yellow #6/sunset yellow FCF aluminum lake, FD&C Blue #2/indigo carmine aluminum lake. (b) 145 mg tablets: polyvinyl alcohol, titanium dioxide, talc, soybean lecithin, xanthan gum.

Evaluation: 48 mg Tricor® tablets were evaluated for weight, hardness, disintegration and dissolution was performed using USP II dissolution apparatus with 900 ml of water with 1% SLS as dissolution medium. 5.0 ml of samples were collected by auto sampler at specified time intervals.

### B. Description and Evaluation of Emulsion.

Description: Emulsion used in the study is viscous fluid containing 15% w/v of drug.

Evaluation: Emulsion was evaluated for total loss on drying by storing a small quantity of emulsion at 40° C. for 2 hr followed by overnight storage at room temperature. Dissolution study of emulsion was also performed by accurately weighing emulsion equivalent to 48 mg of drug in weighing boat and transfer to USP II dissolution jar containing 900 ml of water with 1% SLS as dissolution medium. 5.0 ml of samples were collected by auto sampler at specified time intervals.

### C. Development and Evolution of Tablet Formulations

The aim of the present example is to formulate a fenofibrate emulsion in the form of a solid dosage form. To absorb the emulsion, Neusilin US2 was used, which is a fine powder of magnesium aluminometasilicate (AL₂O₃—MgO-1.7SiO₂-xH2O), an extremely light and porous powder of a fine particle size. Neusilin US2 has a very high oil and water adsorptive capacity due to its large specific surface area. It has shown excellent compressibility and molding capacity as well as dispensability, hence it can be used to form a tablet which after disintegration re-disperses absorbed emulsion. Three formulations (Table 1) were designed for further optimization.

TABLE 1
Exemplary Formulations with IIG limit of inactive ingredients

| | IIG Limit* | Formulations (mg/tablet) | | |
|---|---|---|---|---|
| Ingredients | (mg/tablet) | Nova I | Nova II | Nova III |
| Emulsion 15% w/v (~48 | | 320 | 320 | 320 |
| mg of Fenofibrate) | | | | |
| Neusilin US2, particle | 29.0† | 320 | 160 | 120 |
| size 80 μm | | | | |
| Avicel PH 103 | 1385.3 | 95 | — | — |
| Pre-gelatinized Starch | 435.8 | — | 57.0 | 57 |
| Lactose Monohydrate | 889.42 | — | — | 40 |
| Polyplasdone | 356.82 | 22 | 25 | 25 |
| Povidone K30 | 300 | 7 | — | — |
| Magnesium Sterate | 400.74 | 7 | 1 | 1 |
| Aerosil | 33 | 2 | 1.5 | 1.5 |
| Isopropyl alcohol** | | qs | — | — |
| Water** | | — | qs | qs |
| Total | | 773 | 564.5 | 564.5 |

*Maximum potency of inactive ingredient present in FDA-approved similar (oral tablet) drug product as per “inactive ingredient guide” provided by FDA for tablet dosage form.
http://www.accessdata.fda.gov/scripts/cder/iig/index.cfm
†Value is for magnesium aluminometasilicate
**Not part of the final formulation

### 1. Procedure for Manufacturing Formulation NOVA I:

(a) Add accurately weighed amount of emulsion drop by drop to Neusilin US2 mixed uniformly; (b) Add Avicel PH 103 and ½^{th} of polyplasdone to (I) and granulate with povidone solution in isopropyl alcohol; (c) Dry the wet mass obtained in step II at 40° C. for two hr and pass dry pregranular mass through sieve #30; (d) Blend III with magnesium sterate, ½^{th} of polyplasdone and aerosol; and (e) Compress the final blend using semi-automatic tablet press.

### 2. Procedure for Manufacturing Formulation NOVA II and III

(a) Add accurately weighed amount of emulsion drop by drop to Neusilin US2 and mix uniformly; (b) Add lactose monohydrate and pre-gelatinized starch and ½^{th} of polyplasdone to (I) and granulate with water; (c) Dry the wet mass obtained in step II at 40° C. for two hr and pass the dry pregranular mass through sieve #30; (d) Blend (III) with magnesium sterate, ½^{th} of polyplasdone and aerosol; and (d) Compress the final blend using semi-automatic tablet press.

Evaluation: Granules were evaluated for moisture content, bulk density, tap density and tablets were evaluated for hardness disintegration and dissolution.

### D. Result and Discussion:

Evaluation of Tricor®

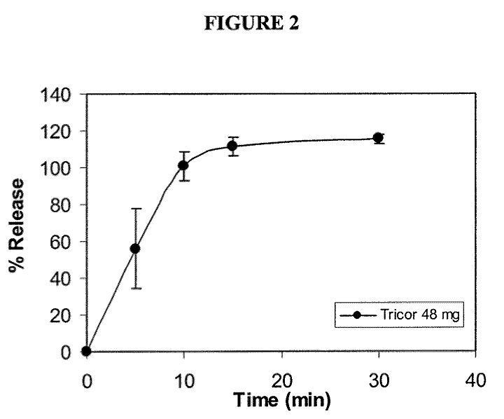

Tricor® tablet disintegrated into a nanospension within 4 min. and the dissolution study shows that almost 100% of drug was released within 20-30 min (FIG. 2).

Formulation Development

Neusilin US2 can take more than two times its weight of emulsion resulting in a powder which was dry and free flowing. In the case of formulation Nova I, it was difficult to archive granulation end point as the amount IPA used was either absorbed by Neusilin US2 or evaporated. This resulted in under-granulation and granules formed were soft with more fines. Tablets formed disintegrated to flakes and disintegration time was more than 10 min. This may be due to the fact that all the ingredients use in this formulation are hydrophobic and may be hindering wetting and the subsequent disintegration process.

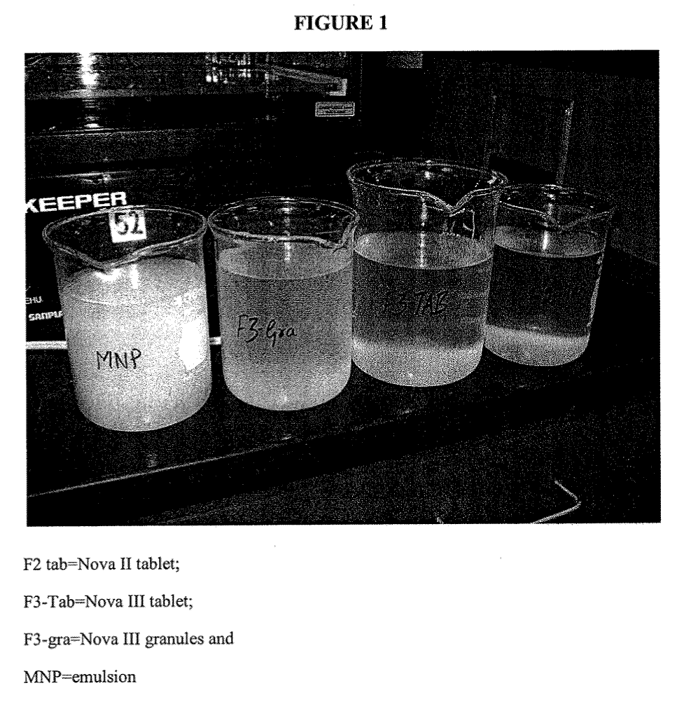

To address these formulation issues, pregelatinized starch (Nova II) was used, which is a well known hydrophilic granulating agent. With a 1:0.5 ratio of emulsion and Neusilin US2 it was possible to achieve granulation end-point, and tables formed from theses granules disintegrated as swollen fragments within 1-2 min (see tables 2 and 3). In spite of fast disintegration, redispersibility of the emulsion from disintegrated swollen fragments was slow and incomplete (See FIG. 1) and floating of oil phase on top of disintegration medium was observed.

TABLE 2
Granular Properties

| Formula- | Moisture | Bulk Density | Tap Density | Compressibility |
|---|---|---|---|---|
| tions | Content (%)* | (BD)g/ml | (TD) g/ml | Index (%)^{a} |
| Nova II | 5.9 | 0.235 | 0.313 | 24.9 |
| Nova III | 5.2 | 0.232 | 0.284 | 18.30 |

Carr's compressibility index (%) = (TD − BD/TD)*100

TABLE 3
Tablet Properties

| Formula- | | Weight | DT^{a} | Hardness^{b} |
|---|---|---|---|---|
| tions | Shape/Dimensions | (gm) | (min) | (kg/cm) |
| Tricor ® | Oval (0.486 in × 0.235 in) | 0.216 | 4.0-5.0 | 3-4 |
| Nova II | Round Double Convex (Dia | 0.282 | 1.0-2.0 | 4-5 |
| | 0.359 in) | | | |
| Nova III | Round Double Convex | 0.282 | 4.0-5.0 | 4-5 |

^{a}In 700 ml of water at 37° C. using USP disintegration apparatus with disks
^{b}Manual spring type hardness tester

Formulation Nova III was formulated with 1:0.375 of emulsion and Neusilin US2 along with lactose and starch. Nova III disintegrated into small granules within 4.0 min (see table 3). Emulsion redispersibility was better with less oil floating than formulation Nova II (See FIG. 1). It was observed that redispersion of emulsion from the granules was relatively better than their tableted form (See FIG. 1).

Redispersibility of emulation from tablets/granules can be evaluated by means of turbidity measurements or particle size analysis of a dispersion. A further optimization of a formulation can be done with additional lactose. Moreover, the development of a capsule dosage form instead of a tablet dosage form can be another parallel option to avoid the effect of compression on redispersibility of the emulsion.

As an emulsion contains both volatile solvents and water, it is necessary to control the loss on drying after the emulsion preparation and before the tablet manufacturing (during shipping and storage). In addition, the drug content of the emulsion may be estimated before granulation. During the granulation process, almost all of the volatile solvents and most of the water will be removed, and the loss on drying of the emulsion alone (not in the form of granules) was 50%. To decide upon the target weight of a tablet equivalent to 48 mg of drug, the drying process was optimized and drug content in the granules was estimated.

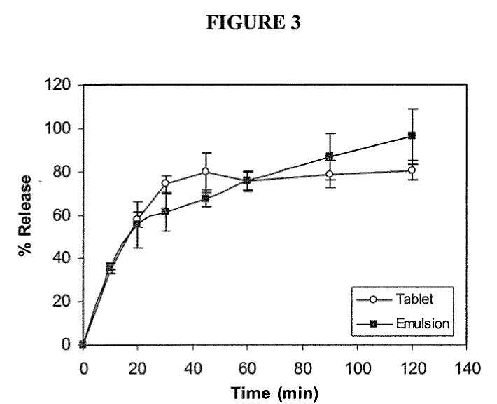

In the present example, each tablet was compressed with a target weight of 0.282 (due to tooling constraint). If the 50% weight of loss of emulsion is factored into dosage design, then theoretically 0.285 mg tablet contains 33.5 mg of drug. From the dissolution results (Table 4 and FIG. 3), it was observed that 75% of the drug was released within 30 min from the tablet, and that the release was not complete even after 2 hr of the dissolution study/However, almost 100% of the drug was released from the emulsion. These results are based upon the theoretical calculation of drug content in both the emulsion and tablet dosage forms.

TABLE 4
Dissolution Data of Tablet and Emulsion

| Time | Nova II tablet | | Emulsion | |
|---|---|---|---|---|
| (min) | (33.5 mg Drug) | Std | (24 mg Drug) | STD |
| 0 | 0.00 | 0.00 | 0.00 | 0.00 |
| 10 | 34.18 | 0.30 | 35.52 | 2.34 |
| 20 | 57.95 | 1.21 | 55.35 | 2.54 |
| 30 | 74.30 | 1.33 | 61.24 | 2.11 |
| 45 | 79.64 | 3.09 | 67.63 | 0.89 |
| 60 | 75.67 | 1.56 | 75.66 | 1.00 |
| 90 | 78.72 | 2.09 | 86.84 | 2.56 |
| 120 | 80.43 | 1.48 | 96.16 | 3.04 |

n = 3

While some embodiments have been illustrated and described, it should be understood that changes and modifications can be made therein in accordance with ordinary skill in the art without departing from the invention in its broader aspects as defined in the following claims.

1. A pharmaceutical formulation comprising:
(a) a multi-phasic pharmaceutical composition comprising at least one active pharmaceutical ingredient (API); and
(b) at least one adsorbent carrier;
wherein the pharmaceutical formulation is a solid dosage form.

2. The pharmaceutical formulation of claim 1, wherein the adsorbent carrier is a clay, a silicate, a cellulose-based polymer, a microsponge, other synthetic polymers, or a mixture of any two or more thereof.

3. The pharmaceutical formulation of claim 2, wherein:
(a) the clay is attapulgite, bentonite, kaolin, perlite, talc, vermiculites, zeolites, or a mixture of any two or more thereof;
(b) the silicate is aluminum silicate, magnesium aluminum silicate, hydrous calcium silicate, colloidal silicon dioxide, magnesium aluminometasilicate, or mixtures of any two or more thereof;
(c) the cellulose-based polymer is carboxymethyl cellulose calcium, carboxymethyl cellulose sodium, cellulose, cellulose acetate, cellulose acetate phthalate, ethyl cellulose, hydroxyethyl cellulose, hydroxypropyl cellulose, hydroxypropyl methylcellulose, hydroxypropyl methylcellulose phthalate, methylcellulose, microcrystalline cellulose, powdered cellulose, or a mixture of any two or more thereof;
(d) the other synthetic polymer is a cross-linked acrylic polymer, a polypropylene, a polyurethane foam, or a mixture of any two or more thereof; or
(e) a combination of any of (a)-(d).

4. The pharmaceutical formulation of claim 3, wherein the adsorbent carrier is calcium carbonate, calcium phosphate dibasic anhydrous, calcium phosphate dibasic dehydrate, calcium phosphate tribasic, calcium sulfate, lactose, magnesium carbonate, magnesium oxide, mannitol, silicon dioxide, sodium starch glycolate, sodium chloride, sorbitol, starch, sucrose, or a mixture of any two or more thereof.

5. The pharmaceutical formulation of claim 4, further comprising a polymeric carrier, a phospholipid carrier, or a mixture of any two or more thereof.

6. The pharmaceutical formulation of claim 5, wherein:
(a) the polymeric carrier is selected from carbomers, croscarmellose sodium, crospovidone, cyclodextrins, β-cyclodextrins, ducosate sodium, hydroxypropyl-β-cyclodextrins, γ-cyclodextrins, polyanionic-β-cyclodextrins, sulfobutylether-7-β-cyclodextrin, methacrylic acid copolymers, poloxamer, polydextrose, polyethylene oxide, polymethacrylate polymers, poly(methacrylic acid-methyl methacrylate), poly(methacrylic acid-ethyl acrylate), ammonio methacrylate copolymer, poly(ethyl acrylate-methylmethacrylate-trimethylammonioethyl methacrylate chloride), poly(ethyl acrylate-methyl methacrylate), polysaccharides, polyvinyl alcohol with an average molecular weight of from about 20,000 to about 200,000 g/mol, polyvinylpyrrolidine/vinylacetate, povidone with an average molecular weight of from about 2,500 to about 300,000 g/mol, sodium starch glycolate, or a mixture of any two or more thereof;
(b) the phospholipid carrier is selected from diphosphatidylglycerol, glycolipids, phosphatidic acid, phosphatidylcholine, phosphatidylethanolamine, phosphatidylglycerol, phosphatidylinositol, phosphatidylserine, sphingomyelin, or a mixture of any two or more thereof; or
(c) any combination thereof.

7. The pharmaceutical formulation of claim 6, wherein the polysaccharide is acacia, alginic acid, carrageenan, ceratonia, chitosan, compressible sugar, confectioner's sugar, confectioner's sugar, dextrates, dextrates, dextrin, dextrin, dextrose, dextrose, fructose, fumaric acid, gelatin, glucose, liquid, glyceryl behenate, guar gum, lactitol, lactose, maltodextrin, maltodextrin, maltose, maltose, mannitol, polydextrose, polymethacrylates, pregelatinized starch, sodium alginate, sodium alginate, sorbitol, starch, pregelatinized starch, sterilizable maize, sucrose, sucrose, sugar spheres, tragacanth, trehalose, xylitol, or a mixture of any two or more thereof.

8. The pharmaceutical formulation of claim 7, further comprising a lubricant.

9. The pharmaceutical formulation of claim 8, wherein the lubricant is magnesium stearate, talc, stearic acid, calcium stearate, zinc stearate, glyceryl palmitostearate, glyceryl behenate, light mineral oil, micronized poloxamers, polyethylene glycol, l-leucine, vegetable oil, or a mixture of any two or more thereof.

10. The pharmaceutical formulation of claim 9, further comprising an antioxidant, a coloring agent, a flavoring agent, a preservative, a sweetener, a volatile oil, or a mixture of any two or more thereof.

11. The pharmaceutical formulation of claim 10, wherein upon deposition in an aqueous medium, the pharmaceutical formulation disintegrates to release an active pharmaceutical ingredient.

12. The pharmaceutical formulation of claim 11, wherein the multiphasic pharmaceutical composition comprises:
(a) at least one active pharmaceutical ingredient (API), wherein the active pharmaceutical ingredient is in a particulate state, a solubilized state, or in both a particulate state and in a solubilized state;
(b) at least one solvent;
(c) at least one non-miscible liquid;
(d) at least one stabilizer; and
(e) water.

13. The pharmaceutical formulation of claim 12, wherein the active pharmaceutical ingredient is selected from agents used in the treatment of AIDS, agents used in treatment of heart disorders, analgesics, anesthetics, anorexiants, anthelmintics, anti-allergic agents, anti-anginal agents, antiarrhythmic agents, anticholinergics, anticoagulants, antidepressants, antidiabetic agents, antidiuretic agents, anti-emetic agents, antiepileptics, anti-fungals, antihistamines, anti-hypertensive agents, anti-inflammatory agents, antimigraine agents, antimuscarinic agents, antimycobacterial agents, antineoplastic agents including, antiparkinsonian agents, antithyroid agents, antiviral agents, astringents, blocking agents, blood products, blood substitutes, cardiac inotropic agents, cardiovascular agents, central nervous system agents, chelating agents, chemotherapy agents, colony stimulating factors, corticosteroids, cough suppressants, dermatological agents, diuretics, dopaminergics, elastase inhibitors, endocrine agents, ergot alkaloids, expectorants, gastrointestinal agents, genitourinary agents, growth hormone releasing hormone, growth hormones, hematological agents, hematopoietic agents, hemostatics, hormones, immunologic agents, immunosuppressants, interleukins, interleukin analogues, lipid regulating agents, luteinizing hormone releasing hormone, muscle relaxants, narcotic antagonists, nutrients, nutritional agents, oncology therapies, organic nitrates, parasympathomimetics, prostaglandins antibiotics, renal agents, respiratory agents, sedatives, sex hormones, stimulants, sympathomimetics, systemic anti-infectives, tactolimuls, thrombolytic agents, thyroid agents, treatments for attention deficit disorder, uterine-active agents, vaccines, vasodilators, xanthines, or mixtures of any two or more thereof.

14. The pharmaceutical formulation of claim 13, wherein the solvent is an alcohol, N-methylpyrrolidinone, methoxypolyethylene glycol, polyethylene glycol, polyethylene oxide, ethoxy diglycol, triacetin, dimethyl sulfoxide, propylene glycol, isopropyl myristate, mono-, di- or tri-glycerides, or a mixture of any two or more thereof.

15. The pharmaceutical formulation of claim 14, wherein the alcohol is benzyl alcohol, ethyl alcohol, methyl alcohol, or a mixture of any two or more thereof.

16. The pharmaceutical formulation of claim 14, wherein the polyethylene glycol has an average molecular weight of about 1000 g/mol or greater, and the methoxypolyethylene glycol has an average molecular weight of about 1000 g/mol or greater.

17. The pharmaceutical formulation of claim 14, wherein the polyethylene glycol has an average molecular weight of from about 1000 g/mol to about 20,000 g/mol, and the methoxypolyethylene glycol has an average molecular weight of from about 1000 g/mol to about 20,000 g/mol.

18. The pharmaceutical formulation of claim 17, wherein the non-miscible liquid is a fatty acid, a medium chain glyceride, a long chain glyceride, an ethyl ester of a fatty acid, a propylene glycol fatty acid ester, a sorbitan fatty acid ester, a polyglyceryl fatty acid ester, a glyceryl mono-, di-, or tri-caprylic acid ester; a glyceryl mono-, di-, or tri-capric acid esters; or a mixture of any two or more thereof.

19. The pharmaceutical formulation of claim 18, wherein the non-miscible liquid is selected from vegetable oils, nut oils, fish oils, lard oil, mineral oils, squalane, tricaprylin (1,2,3-trioctanoyl glycerol), and mixtures of any two or more thereof.

20. The pharmaceutical formulation of claim 19, wherein the non-miscible liquid is almond oil (sweet), apricot seed oil, borage oil, canola oil, coconut oil, corn oil, cotton seed oil, fish oil, jojoba bean oil, lard oil, linseed oil (boiled), macadamia nut oil, medium chain triglycerides, mineral oil, olive oil, peanut oil, safflower oil, sesame oil, soybean oil, sunflower seed oil, wheat germ oil, mineral oil (light), DL-α-tocopherol, ethyl oleate, ethyl linoleate, glyceryl behenate, glyceryl monooleate, glyceryl monostearate, glyceryl palmitostearate, linoleic acid, linolenic acid, oleic acid, palmitostearic acid, peppermint oil, polyglyceryl oleate, propylene glycol monolaureate, propylene glycol dilaureate, sorbitan monolaurate, sorbitan monooleate, sorbitan monopalmitate, sorbitan monostearate, sorbitan trioleate, stearic acid, tetraglyceryl monooleate, or a mixture of any two or more thereof.

21. The pharmaceutical formulation of claim 20, wherein the stabilizer is selected from non-phospholipid surfactants, non-phenol polyethylene glycol ethers, sorbitan esters, polyethylene glycol esters, block polymers, acrylic polymers, ethoxylated fatty acids, ethoxylated alcohols, ethoxylated fatty acid esters, monoglycerides, silicon-based surfactants, polysorbates, tergitols, sugar fatty acid ester; a sucrose mono-, di-, or tri-fatty acid ester; a polyoxyethylene castor oil compound; a polyoxyethylene sorbitan fatty acid ester; a polyoxyethylene mono- or di-fatty acid ester; a polyoxyethylene alkyl ether; a glyceryl mono-, di-, or tri-fatty acid ester; a mixtures of polyoxyethylene mono- or di-ester of a C8-C22 fatty acid; a glyceryl mono-, di-, or tri-ester of a C8-C22 fatty acid, or a mixture of any two or more thereof.

22. The pharmaceutical formulation of claim 21, wherein the stabilizer is selected from ARLACEL™, BRIJ™, Cremophore RH-40, glycerin monostearate, PEMULEN™, PLURONIC™, polyethylene glycol stearate, polyoxyl 35 castor oil, polyoxyl 40 hydrogenated castor oil, polyoxyl 60 hydrogenated castor oil, polysorbate 20, polysorbate 40, polysorbate 60, polysorbate 80, polyoxyl 40 stearate, polyoxyl 40 oleate, polyoxyl 20 cetostearyl ether, polyoxyl 10 oleyl ether, sodium dioctyl sulfosuccinate, sodium lauryl sulfate, SPAN™, TERGITOL™ NP-40, TERGITOL™ NP-70, DL-α-tocopheryl polyethylene glycol succinate, TWEEN™20, TWEEN™60, TWEEN™80, or a mixture of any two or more thereof.

23. The pharmaceutical formulation of claim 22, wherein upon deposition in an aqueous medium, the pharmaceutical formulation disintegrates to release the active pharmaceutical ingredient.

24. A pharmaceutical formulation comprising:
(a) a multi-phasic pharmaceutical composition comprising at least one active pharmaceutical ingredient (API);
(b) at least one adsorbent carrier; and
(c) at least one disintegrant;
wherein the pharmaceutical formulation is a solid dosage form.

25. The pharmaceutical formulation of claim 24 comprising two different APIs in a single solid dosage form.

26. The pharmaceutical formulation of claim 25, wherein the active pharmaceutical ingredient or the multi-phasic pharmaceutical composition is present at about 0.1 to about 90 wt %.

27. The pharmaceutical formulation of claim 26, wherein the solid dosage form is a capsule or tablet.

28. A method of preparing a pharmaceutical formulation comprising:
(a) mixing at least one active pharmaceutical ingredient, at least one solvent, at least one stabilizer, and at least one non-miscible liquid to form a first mixture;
(b) emulsifying the first mixture with water to form a multi-phasic pharmaceutical composition; and
(c) mixing the emulsified first mixture with an adsorbent carrier to form a solid dosage form;
wherein the active pharmaceutical ingredient is present in the multi-phasic pharmaceutical composition in solubilized state, a particulate state, or in both a particulate state and a solubilized state.

29. The method of claim 28, further comprising granulating the solid dosage form and pressing the solid dosage form into a capsule or tablet.

30. The method of claim 29, wherein at least one active pharmaceutical ingredient comprises from about 0.1 to about 90 wt % of the capsule or tablet.

31. The method of claim 30, wherein the multi-phasic pharmaceutical composition comprises globules of the non-miscible liquid and the globules have a diameter of less than about 10 μm.

32. The method of claim 31, wherein the globules have a diameter of less than about 9 microns, less than about 8 microns, less than about 7 microns, less than about 6 microns, less than about 5 microns, less than about 4 microns, less than about 3 microns, less than about 2 microns, less than about 1000 nm, less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

33. The method of claim 32, wherein an average diameter of the particles of the particulate state is less than about 1 micron.

34. The method of claim 32, wherein the average diameter is less than about 900 nm, less than about 800 nm, less than about 700 nm, less than about 600 nm, less than about 500 nm, less than about 400 nm, less than about 300 nm, less than about 290 nm, less than about 280 nm, less than about 270 nm, less than about 260 nm, less than about 250 nm, less than about 240 nm, less than about 230 nm, less than about 220 nm, less than about 210 nm, less than about 200 nm, less than about 190 nm, less than about 180 nm, less than about 170 nm, less than about 160 nm, less than about 150 nm, less than about 140 nm, less than about 130 nm, less than about 120 nm, less than about 110 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.
